(19) United States
(12) Patent Application Publication
(10) Pub. No.: US 2024/0000299 A1
(43) Pub. Date: Jan. 4, 2024

(54) IMAGE PROCESSING APPARATUS, IMAGE PROCESSING METHOD, AND PROGRAM

(71) Applicant: FUJIFILM Corporation, Tokyo (JP)

(72) Inventor: Misaki GOTO, Kanagawa (JP)

(73) Assignee: FUJIFILM Corporation, Tokyo (JP)

(21) Appl. No.: 18/468,748

(22) Filed: Sep. 18, 2023

Related U.S. Application Data
(63) Continuation of application No. PCT/JP2022/010892, filed on Mar. 11, 2022.

(30) Foreign Application Priority Data
Mar. 22, 2021 (JP) 2021-047136

(51) Int. Cl. A61B 1/04; A61B 1/00; G06T 7/00; G06T 7/50; G06T 7/60

(52) U.S. Cl. CPC A61B 1/04; A61B 1/00009; G06T 7/0012; G06T 7/50; G06T 7/60; G06V 2201/07; G06T 2207/10068

(57) ABSTRACT

An image processing apparatus, an image processing method, and a program capable of acquiring correct scope information and acquiring accurate depth information from an intraluminal image. In image processing apparatus (14) including a processor, the processor performs image acquisition processing of acquiring a time-series intraluminal image captured by a scope of an endoscope; scope information acquisition processing of acquiring scope information relating to a change of the scope; landmark recognition processing of recognizing a landmark in the intraluminal image; scope information correction processing of correcting the scope information using information relating to the landmark recognized in the landmark recognition processing; and depth information acquisition processing of acquiring depth information of the intraluminal image using the intraluminal image and the scope information corrected in the scope information correction processing.

### CROSS-REFERENCE TO RELATED APPLICATIONS

The present application is a Continuation of PCT International Application No. PCT/JP2022/010892 filed on Mar. 11, 2022 claiming priority under 35 U.S.C § 119(a) to Japanese Patent Application No. 2021-047136 filed on Mar. 22, 2021. Each of the above applications is hereby expressly incorporated by reference, in its entirety, into the present application.

### BACKGROUND OF THE INVENTION

### 1. Field of the Invention

The present invention relates to an image processing apparatus, an image processing method, and a program, and more particularly relates to an image processing apparatus, an image processing method, and a program of acquiring depth information of an intraluminal image.

### 2. Description of the Related Art

In observation performed using an endoscope system (endoscope apparatus), a technique of displaying the position of an endoscopic scope of the endoscope system, the shape of a lumen, and the position of a lesion in association with each other is known. This technique can effectively assist a user in comprehensively observing a lumen (for example, a large intestine) that is an observation target. To geometrically recognize the current position of the endoscopic scope, the shape of the lumen, and the position of the lesion, it is necessary to accurately estimate the depth from a camera included in a tip part of the scope to a target object.

WO2007/139187A proposes a technique of acquiring distance information (depth information) based on brightness information of an endoscopic image and constructing a three-dimensional image. Also, WO2007/139187A describes a technique of acquiring a change amount in an axial direction and a change amount in a circumferential direction of the endoscopic scope by a motion detection sensor, and correcting a developed image based on the acquired change amounts.

### SUMMARY OF THE INVENTION

In the above-described technique, to acquire correct depth information, it is necessary to correctly acquire scope information relating to a change of the endoscopic scope (for example, an insertion length of the scope into a lumen, and a bending angle and a rotation amount of the scope in the lumen).

However, since the lumen of the observation target of the endoscope system is a non-rigid body, scope information of an actual measurement value acquired by a sensor or the like described in WO2007/139187A is not correct in some cases. That is, the scope information acquired by the sensor or the like may be different from the actual relative change amount of the scope in the lumen. As described above, when the scope information cannot be correctly acquired, the depth information acquired using the scope information is also incorrect.

The present invention is made in view of the situation, and an object of the invention is to provide an image processing apparatus, an image processing method, and a program capable of acquiring correct scope information and acquiring accurate depth information from an intraluminal image.

An image processing apparatus according to an aspect of the present invention to attain the above-described object is an image processing apparatus including a processor. The processor performs image acquisition processing of acquiring a time-series intraluminal image captured by a scope of an endoscope; scope information acquisition processing of acquiring scope information relating to a change of the scope; landmark recognition processing of recognizing a landmark in the intraluminal image; scope information correction processing of correcting the scope information using information relating to the landmark recognized in the landmark recognition processing; and depth information acquisition processing of acquiring depth information of the intraluminal image using the intraluminal image and the scope information corrected in the scope information correction processing.

With this aspect, the landmark in the intraluminal image is recognized, and the scope information is corrected using the information relating to the recognized landmark. Accordingly, accurate depth information of the intraluminal image can be acquired based on the accurate scope information.

Preferably, with reference to a position of the scope at a time T, the scope information acquisition processing acquires a change amount of an insertion length of the scope and change amounts relating to bending and rotation of the scope at a time T+α.

Preferably, the scope information acquisition processing acquires information relating to an insertion length of the scope and bending and a rotation of the scope from an operation of an operation section of the scope.

Preferably, the landmark recognition processing recognizes a temporal change of a correspondence point of the landmark, and the scope information correction processing corrects the scope information using the temporal change of the correspondence point.

Preferably, the landmark recognition processing outputs recognition reliability of the recognized landmark, and the scope information correction processing determines whether to execute the correction of the scope information based on the recognition reliability, and performs the correction based on a result of the determination.

With this aspect, the recognition reliability of the landmark is output, and it is determined whether to execute the correction of the scope information based on the recognition reliability. Accordingly, accurate correction can be executed and accurate distance information can be acquired.

Preferably, the scope information correction processing outputs a correction value obtained from the information relating to the landmark, determines whether to execute the correction based on the correction value, and performs the correction based on a result of the determination.

With this aspect, the correction value is output from the information relating to the landmark, and it is determined whether to execute the correction based on the output correction value. Accordingly, accurate correction can be executed and accurate distance information can be acquired.

Preferably, the processor performs display control processing of displaying geometric information relating to a lumen on a display unit based on the depth information acquired in the depth information acquisition processing.

With this aspect, since the geometric information relating to the lumen is displayed on the display unit based on the acquired depth information, correct geometric information relating to the scope can be provided to the user.

Preferably, the geometric information is at least one of a shape of the lumen, a position of a lesion, a position of the scope, or a position of a treatment tool.

An image processing method according to another aspect of the present invention is an image processing method using an image processing apparatus including a processor. The method, performed by the processor, includes an image acquisition step of acquiring a time-series intraluminal image captured by a scope of an endoscope; a scope information acquisition step of acquiring scope information relating to a change of the scope; a landmark recognition step of recognizing a landmark in the intraluminal image; a scope information correction step of correcting the scope information using information relating to the landmark recognized in the landmark recognition step; and a depth information acquisition step of acquiring depth information of the intraluminal image using the intraluminal image and the scope information corrected in the scope information correction step.

A program according to still another aspect of the present invention is a program causing an image processing method to be executed using an image processing apparatus including a processor. The program causing the processor to execute an image acquisition step of acquiring a time-series intraluminal image captured by a scope of an endoscope; a scope information acquisition step of acquiring scope information relating to a change of the scope; a landmark recognition step of recognizing a landmark in the intraluminal image; a scope information correction step of correcting the scope information using information relating to the landmark recognized in the landmark recognition step; and a depth information acquisition step of acquiring depth information of the intraluminal image using the intraluminal image and the scope information corrected in the scope information correction step.

According to the present invention, since the landmark in the intraluminal image is recognized, and the scope information is corrected using the information relating to the recognized landmark, accurate depth information of the intraluminal image can be acquired based on accurate scope information.

### BRIEF DESCRIPTION OF THE DRAWINGS

FIG. 1 is a view schematically illustrating an endoscopic image and a depth image obtained from the endoscopic image;

FIG. 2 is a view illustrating geometric information relating to a large intestine and a corresponding endoscopic image;

FIG. 3 is a diagram explaining acquisition of depth information from an endoscopic image and scope information;

FIG. 4 is a schematic diagram illustrating the entire configuration of an endoscope system including an image processing apparatus;

FIG. 5 is a block diagram illustrating an embodiment of the image processing apparatus;

FIG. 6 is a diagram explaining acquisition of scope information;

FIG. 7 is a diagram explaining acquisition of scope information;

FIG. 8 is a view explaining an example of a landmark;

FIG. 9 is a diagram explaining information relating to a landmark;

FIG. 10 is a diagram explaining acquisition of a depth image from corrected scope information and an intraluminal image;

FIG. 11 is a view illustrating an example of geometric information of a lumen displayed on a display unit;

FIG. 12 is a diagram explaining a flow of acquisition of depth information;

FIG. 13 is a diagram explaining a flow of acquisition of depth information;

FIG. 14 is a flowchart presenting an image processing method; and

FIG. 15 is a flowchart presenting an image processing method.

### DESCRIPTION OF THE PREFERRED EMBODIMENTS

Hereinafter, preferred embodiments of an image processing apparatus, an image processing method, and a program according to the present invention will be described with reference to the accompanying drawings.

Acquisition of Depth Information from Endoscopic Image

First, acquisition of depth information from an endoscopic image will be described.

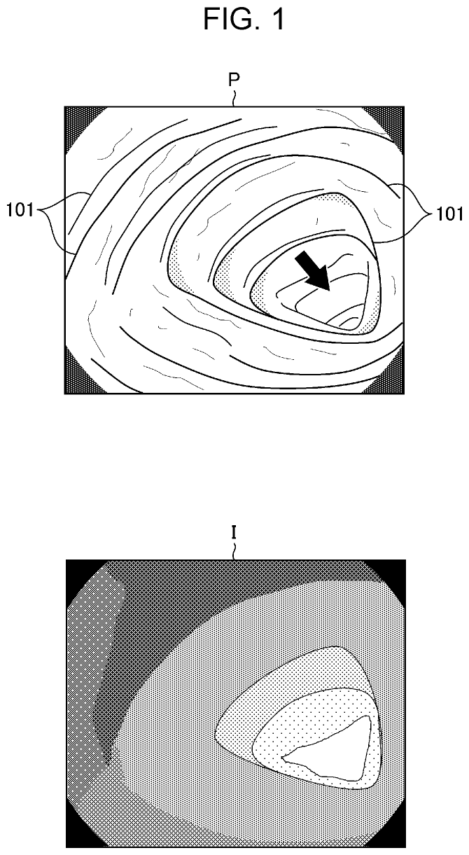

FIG. 1 is a view schematically illustrating an endoscopic image and a depth image obtained from the endoscopic image.

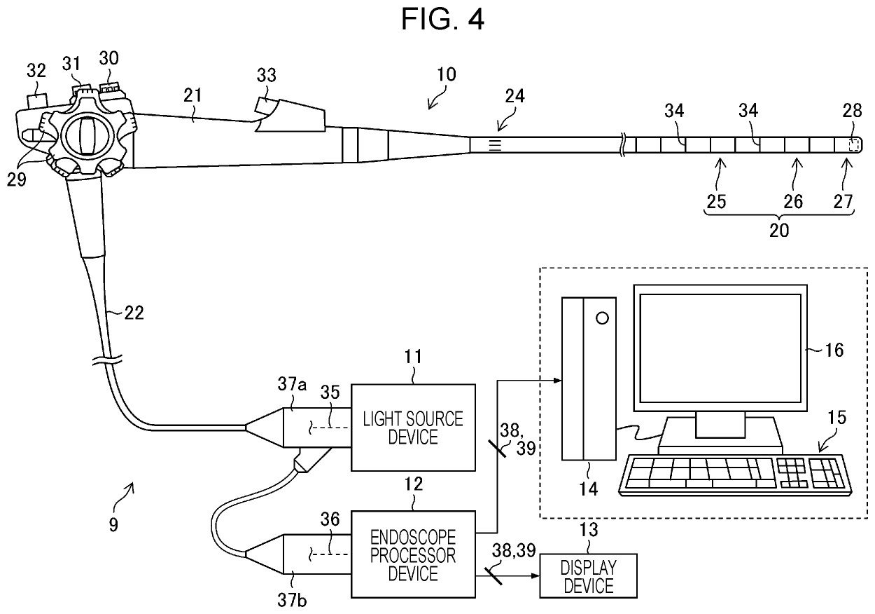

FIG. 1 illustrates an intraluminal image P that is an example of an endoscopic image acquired by an endoscope system 9 (FIG. 4). The intraluminal image P is, for example, an image obtained by image-capturing the inside of a large intestine. In the intraluminal image P, a fold 101 in the large intestine is presented, and a tubular shape continues in an arrow direction. A depth image I is an image having depth information corresponding to the intraluminal image P. The depth image I has information relating to a depth (distance) from a camera (for example, an imaging element 28 (FIG. 4)). In the depth image I, depth information is presented in a heat map form. Note that the depth image I is presented in a simplified manner, and specifically, seven regions having mutually different items of depth information are presented. Note that, in the depth image I, the depth information may be actually presented in a heat map form with finer regions, and for example, a different item of depth information may be presented every pixel. Also, in this example, as an example of an intraluminal image, a case where the large intestine is observed by the endoscope system 9 will be described, but the example of the intraluminal image is not limited thereto. The intraluminal image may be an image obtained by image-capturing another luminal organ.

Normally, the above-described depth image I having the depth information is acquired using images at a plurality of viewpoints whose relative positional relationship is known, such as a stereo camera. However, since the endoscope system 9 includes a monocular camera, when the depth information is acquired, it is necessary to acquire the depth information based on an endoscopic image acquired by the monocular camera.

For example, a document (Daniel Freedman et al., “Detecting Deficient Coverage in Colonoscopies”, CVPR2020, https://arxiv.org/pdf/2001.08589.pdf) describes a technique of acquiring a depth image having depth information from an endoscopic image acquired by a monocular camera using a recognizer constituted of a convolutional neural network (CNN).

When the depth information is acquired using only the endoscopic image captured by the above-described monocular camera, a relative depth is estimated while the movement amount of an endoscopic scope 10 (see FIG. 4) between adjacent frames is estimated. In this case, since the organ is a non-rigid body, the lumen shape changes every frame, and an error may occur in the depth information.

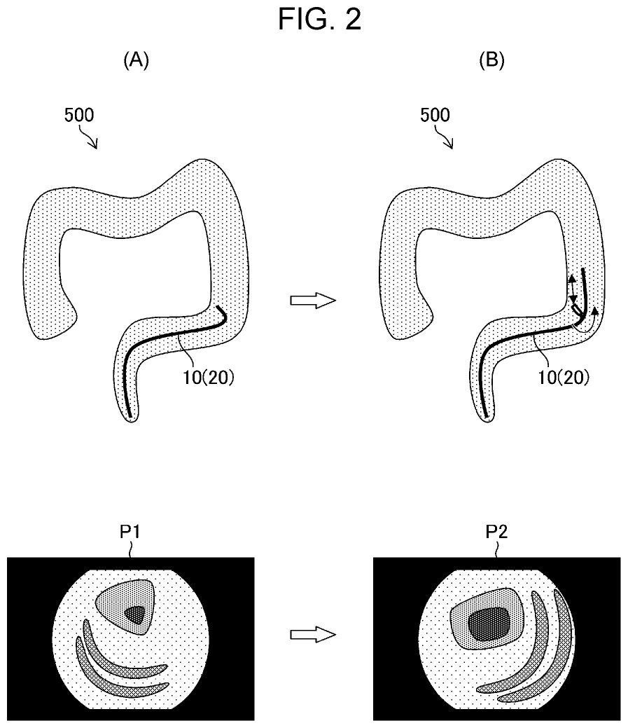

FIG. 2 is a view illustrating geometric information relating to a large intestine and a corresponding endoscopic image.

Geometric information 500 relating to the large intestine indicates the current position of an insertion section 20 of the endoscopic scope 10. Also, an endoscopic image acquired at the position of the endoscopic scope 10 indicated in the geometric information 500 of the large intestine is presented.

As illustrated in FIG. 2(A), an intraluminal image P1 is acquired at the position of the insertion section 20 indicated in the geometric information 500. As illustrated in FIG. 2(B), when the endoscopic scope 10 is translated, bent, and rotated from the position indicated in FIG. 2(A), an intraluminal image P2 is acquired. The viewpoint and the lumen shape change between the intraluminal image P1 and the intraluminal image P2 due to the movement of the endoscopic scope 10 and the influence of the observation target (large intestine) that is a non-rigid body. In such a case, when depth information is acquired using only an intraluminal image captured by the above-described monocular camera, an error may occur in the depth information.

As described above, since the observation target is a non-rigid body, in the case where the depth information is acquired from the intraluminal image, accuracy may be decreased when the depth information is acquired using only the intraluminal image.

Acquisition of Depth Information from Endoscopic Image and Scope Information

To suppress the above-described decrease in the accuracy of the depth information, it is considered that an actual measurement value of scope information is acquired, and depth information is acquired from the scope information and an endoscopic image.

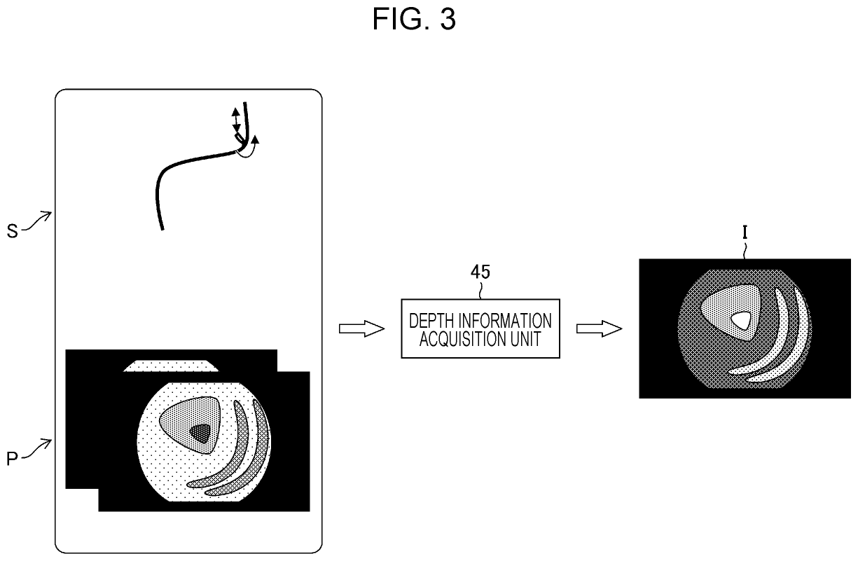

FIG. 3 is a diagram explaining acquisition of depth information from an endoscopic image and scope information.

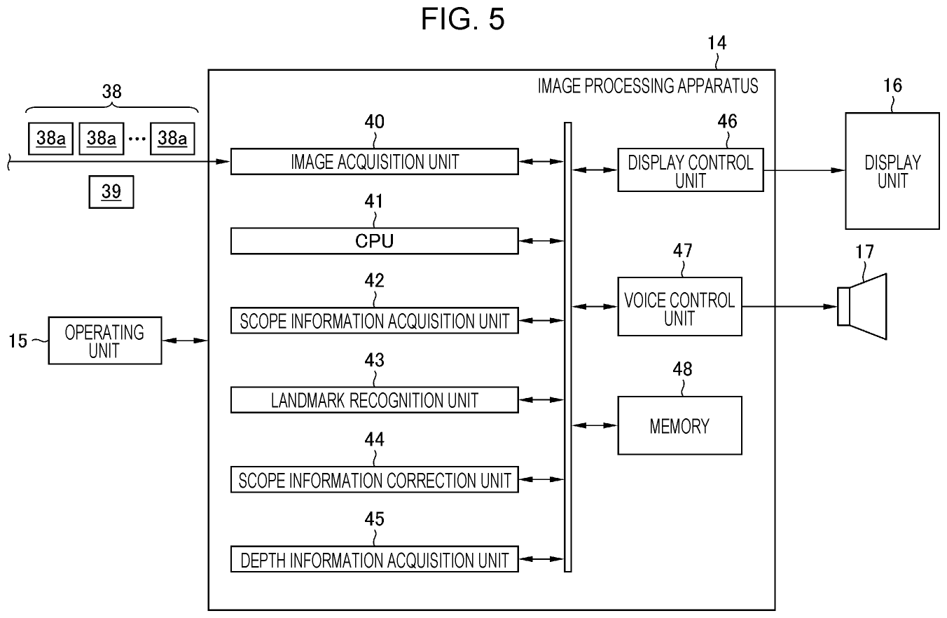

As illustrated in FIG. 3, scope information S and an intraluminal image P are input to a depth information acquisition unit 45 (see FIG. 5), and a depth image I having depth information is output. Here, the scope information S is information indicating a change of the endoscopic scope 10, and is an actual measurement value. For example, the scope information S is an insertion length of the endoscopic scope 10, and a bending angle and a rotation amount of the endoscopic scope 10. Also, the depth information acquisition unit 45 is a learned model constituted of a CNN, and learning is performed to output a depth image I in response to input of scope information S and an intraluminal image P. As described above, by inputting the scope information S of the actual measurement value to the depth information acquisition unit 45, calibration of the movement change amount of the endoscopic scope 10 (specifically, the position of the imaging element 28) can be performed, and more correct depth information can be acquired.

### Correction of Scope Information

Next, correction of the scope information S of the actual measurement value described above will be described.

The scope information S is an actual measurement value, and basically, an error does not occur with respect to the change amount of the endoscopic scope 10 in the actual lumen. However, since the observation target is a non-rigid body organ, the scope information S obtained as an actual measurement value and the relative change amount by which the scope has changed in the lumen do not coincide with each other in some cases. In such a case, the accuracy of the depth image I output from the depth information acquisition unit 45 based on the scope information S and the intraluminal image P may be low.

Here, by correcting the scope information S that is an actual measurement value using information relating to a landmark in the endoscopic image, information closer to the relative change amount of the endoscopic scope 10 in the lumen can be acquired. Thus, the present invention proposes a method of correcting scope information S of an actual measurement value using a landmark in an endoscopic image, and acquiring more correct depth information based on the corrected scope information T.

### First Embodiment

### Entire Configuration of Endoscope System Including Image Processing Apparatus

FIG. 4 is a schematic diagram illustrating the entire configuration of an endoscope system including an image processing apparatus.

As illustrated in FIG. 4, an endoscope system 9 includes an endoscopic scope 10 that is an electronic endoscope, a light source device 11, an endoscope processor device 12, a display device 13, an image processing apparatus 14, an operating unit 15, and a display unit 16.

The endoscopic scope 10 captures a time-series endoscopic image including a subject image, and is, for example, a lower or upper gastrointestinal tract scope. The endoscopic scope 10 has an insertion section 20 that is inserted into a subject (for example, stomach) and has a distal end and a proximal end, a hand operation section 21 that is connected to the proximal end side of the insertion section 20 and that is gripped by a doctor, who is an operator, to perform various operations, and a universal cord 22 that is connected to the hand operation section 21. Also, a rotation scale 24 is provided at the endoscopic scope 10. The user can obtain the rotation amount in a circumferential direction of the endoscopic scope 10 by reading the rotation scale 24. Here, the circumferential direction is a circumferential direction of a circle centered on the axis of the endoscopic scope 10.

The insertion section 20 is formed in an elongated shape with a small diameter as a whole. The insertion section 20 is constituted by continuously providing, in order from the proximal end side to the distal end side thereof, a soft part 25 having flexibility, a bending part 26 bendable by an operation of the hand operation section 21, and a tip part 27 in which an imaging optical system (objective lens) (not illustrated), an imaging element 28, and the like, are incorporated. Note that, a length scale 34 indicating an insertion length (push-in amount) of the insertion section 20 is provided at the insertion section 20. The user can obtain the insertion length of the insertion section 20 by reading the length scale 34.

The imaging element 28 is a complementary metal oxide semiconductor (CMOS) type imaging element or a charge coupled device (CCD) type imaging element. Image light of a part to be observed is incident on an imaging surface of the imaging element 28 via an observation window (not illustrated) opened in a distal end surface of the tip part 27 and the objective lens (not illustrated) disposed behind the observation window. The imaging element 28 captures the image light of the part to be observed incident on the imaging surface thereof (converts the image light into an electric signal), and outputs an imaging signal. That is, endoscopic images are sequentially captured by the imaging element 28. Note that the endoscopic images are acquired as a moving image 38 and a still image 39 (described later).

The hand operation section 21 is provided with various operation members that are operated by the doctor (user). Specifically, the hand operation section 21 is provided with two types of bending operation knobs 29 used for bending operations of the bending part 26, an air/water supply button 30 for an air/water supply operation, and a suction button 31 for a suction operation. Also, the hand operation section 21 is provided with a still image capturing instruction portion 32 for giving an instruction to capture a still image 39 of the part to be observed, and a treatment tool lead-in port 33 for inserting a treatment tool (not illustrated) into a treatment tool insertion passage (not illustrated) inserted through the insertion section 20.

The universal cord 22 is a connection cord for connecting the endoscopic scope 10 to the light source device 11. The universal cord 22 incorporates a light guide 35, a signal cable 36, and a fluid tube (not illustrated) that are inserted through the insertion section 20. Also, a connector 37a that is connected to the light source device 11 and a connector 37b that is branched from the connector 37a and connected to the endoscope processor device 12 are provided at an end portion of the universal cord 22.

By connecting the connector 37a to the light source device 11, the light guide 35 and the fluid tube (not illustrated) are inserted into the light source device 11. Accordingly, necessary illumination light, water, and gas are supplied from the light source device 11 to the endoscopic scope 10 via the light guide 35 and the fluid tube (not illustrated). As a result, illumination light is emitted from an illumination window (not illustrated) in the distal end surface of the tip part 27 toward the part to be observed. Also, in accordance with a pressing operation of the above-described air/water supply button 30, a gas or water is ejected from an air/water supply nozzle (not illustrated) in the distal end surface of the tip part 27 toward the observation window (not illustrated) in the distal end surface.

By connecting the connector 37b to the endoscope processor device 12, the signal cable 36 and the endoscope processor device 12 are electrically connected to each other. Accordingly, via the signal cable 36, an imaging signal of the part to be observed is output from the imaging element 28 of the endoscopic scope 10 to the endoscope processor device 12, and a control signal is output from the endoscope processor device 12 to the endoscopic scope 10.

The light source device 11 supplies illumination light to the light guide 35 of the endoscopic scope 10 via the connector 37a. As the illumination light, light in various wavelength ranges according to the observation purpose, such as white light (light in a white wavelength range or light in a plurality of wavelength ranges), light in one or a plurality of specific wavelength ranges, or a combination thereof, is selected.

The endoscope processor device 12 controls the operation of the endoscopic scope 10 via the connector 37b and the signal cable 36. Also, the endoscope processor device 12 generates an image (also referred to as a “moving image 38”) consisting of time-series frame images 38a including a subject image based on imaging signals acquired from the imaging element 28 of the endoscopic scope 10 via the connector 37b and the signal cable 36. Further, when the still image capturing instruction portion 32 is operated at the hand operation section 21 of the endoscopic scope 10, the endoscope processor device 12 sets one frame image 38a in the moving image 38 as a still image 39 corresponding to the timing of the capturing instruction in parallel with generation of the moving image 38.

The moving image 38 and the still image 39 are endoscopic images obtained by image-capturing the inside of a subject, that is, the inside of a living body. Further, when the moving image 38 and the still image 39 are images obtained with light (special light) in the above-described specific wavelength range, both are special light images. Then, the endoscope processor device 12 outputs the generated moving image 38 and still image 39 to the display device 13 and the image processing apparatus 14.

The endoscope processor device 12 may generate (acquire) a special light image having information of the above-described specific wavelength range based on a normal light image obtained with the above-described white light. In this case, the endoscope processor device 12 functions as a special light image acquisition unit. The endoscope processor device 12 obtains a signal in the specific wavelength range by performing calculation based on color information of red, green, and blue (RGB), or cyan, magenta, and yellow (CMY) included in the normal light image.

Alternatively, for example, the endoscope processor device 12 may generate a feature amount image such as a known oxygen saturation image based on at least one of a normal light image obtained with the above-described white light or a special light image obtained with the above-described light (special light) in the specific wavelength range. In this case, the endoscope processor device 12 functions as a feature amount image generation unit. Note that any of the moving image 38 or the still image 39 including the in-vivo image, the normal light image, the special light image, and the feature amount image described above is an endoscopic image obtained by image-capturing a human body or by imaging a measurement result for the purpose of diagnosis or inspection using an image.

The display device 13 is connected to the endoscope processor device 12, and functions as the display unit 16 that displays the moving image 38 and the still image 39 input from the endoscope processor device 12. The doctor (user) performs an advancing/retracting operation or the like of the insertion section 20 while checking the moving image 38 displayed on the display device 13, when finding a lesion or the like in the part to be observed, operates the still image capturing instruction portion 32 to execute still image capturing of the part to be observed, and performs a treatment such as diagnosis or biopsy. Note that the moving image 38 and the still image 39 are similarly displayed on the display unit 16 connected to the image processing apparatus 14 (described later). Also, when the moving image 38 and the still image 39 are displayed on the display unit 16, notification display (described later) is also performed together. Thus, the user preferably performs diagnosis or the like while viewing the display on the display unit 16.

### Image Processing Apparatus

FIG. 5 is a block diagram illustrating an embodiment of the image processing apparatus 14. The image processing apparatus 14 sequentially acquires time-series endoscopic images, and displays the endoscopic images and geometric information relating to the lumen on the display unit 16. The image processing apparatus 14 is constituted of, for example, a computer. The operating unit 15 includes buttons provided at the hand operation section 21 of the endoscopic scope 10 in addition to a keyboard, a mouse, and the like, connected to the computer in a wired or wireless manner, and various monitors such as a liquid crystal monitor connectable to the computer are used as the display unit 16.

The image processing apparatus 14 is composed of an image acquisition unit 40, a central processing unit (CPU) 41, a scope information acquisition unit 42, a landmark recognition unit 43, a scope information correction unit 44, a depth information acquisition unit 45, a display control unit 46, a voice control unit 47, and a memory 48. The processing of each unit is implemented by one or more processors. Here, the processor may be constituted of the CPU 41, or may be constituted of one or more CPUs (not illustrated).

The CPU 41 operates based on various programs including an operation system and an endoscopic image processing program stored in the memory 48, generally controls the image acquisition unit 40, the scope information acquisition unit 42, the landmark recognition unit 43, the scope information correction unit 44, the depth information acquisition unit 45, the display control unit 46, and the voice control unit 47, and functions as a portion of each of these units.

The image acquisition unit 40 performs image acquisition processing to sequentially acquire time-series endoscopic images. The image acquisition unit 40 acquires time-series endoscopic images including a subject image from the endoscope processor device 12 (FIG. 4) using an image input/output interface (not illustrated) connected to the endoscope processor device 12 in a wired or wireless manner. In this example, the moving image 38 captured by the endoscopic scope 10 is acquired. Also, when the above-described still image 39 is captured while the moving image 38 is being captured by the endoscopic scope 10, the image acquisition unit 40 acquires the moving image 38 and the still image 39 from the endoscope processor device 12. In this example, an intraluminal image P (FIG. 1) obtained by image-capturing a large intestine will be described as an example of an endoscopic image.

The memory 48 includes a flash memory, a read-only memory (ROM), a random access memory (RAM), a hard disk device, and the like. The flash memory, the ROM, and the hard disk device are nonvolatile memories that store the operation system, various programs such as the endoscopic image processing program, the captured still image 39, and the like. Also, the RAM is a volatile memory that can read/write data at high speed and that functions as an area for temporarily storing the various programs stored in the nonvolatile memories and as a work area for the CPU 41.

The scope information acquisition unit 42 performs scope information acquisition processing to acquire scope information relating to a change of the endoscopic scope 10. The scope information is information indicating an operation of the insertion section 20 of the endoscopic scope 10. Specifically, the scope information includes an insertion length indicating the length by which the insertion section 20 of the endoscopic scope 10 is pushed into a lumen, a bending angle indicating the bending of the bending part 26, and a rotation amount indicating the rotation of the endoscopic scope 10 in the circumferential direction. The scope information S can be acquired by actual measurement, and the scope information acquisition unit 42 can acquire the scope information S by various methods. For example, when the scope information acquisition unit 42 acquires the insertion length, the insertion length may be acquired by image-capturing the length scale 34 provided at the insertion section 20 by a camera, or the insertion length may be acquired by a sensor (not illustrated) provided together with the length scale 34. Also, for example, when the scope information acquisition unit 42 acquires the bending angle, the bending angle may be acquired based on the rotation amount of the bending operation knob 29, or the bending angle may be acquired by a sensor (not illustrated) provided at the bending part 26. Further, for example, when the scope information acquisition unit 42 acquires the rotation amount, the rotation scale 24 provided at the endoscopic scope 10 may be image-captured by a camera, and the read rotation amount may be acquired, or the rotation amount in the circumferential direction of the endoscopic scope 10 may be acquired by a gyro sensor (not illustrated) incorporated in the hand operation section 21.

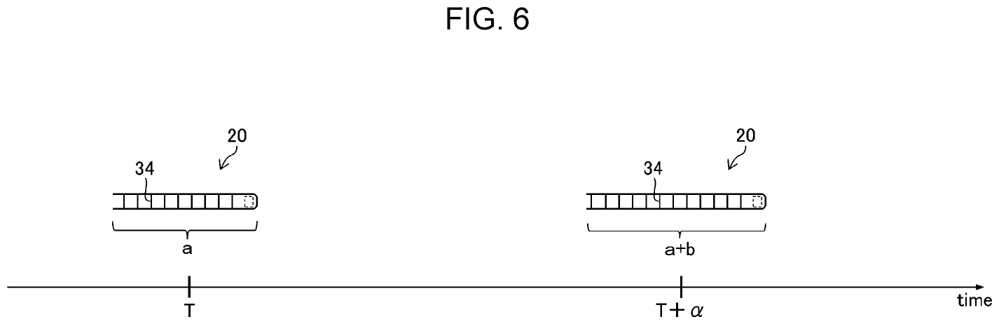

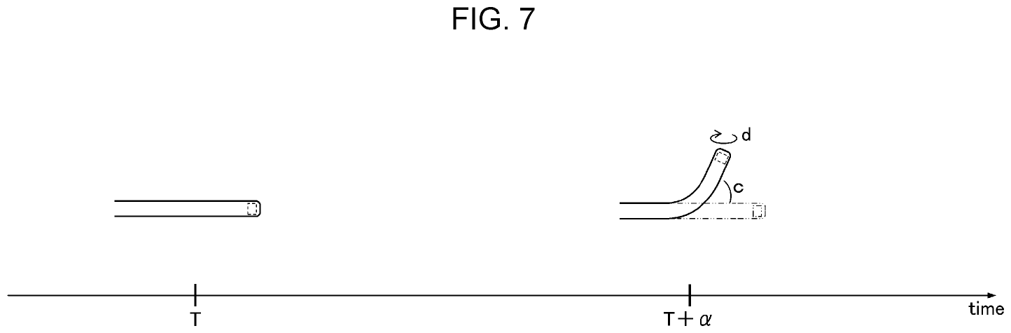

FIGS. 6 and 7 are views explaining acquisition of scope information S. FIG. 6 is a diagram explaining acquisition of the insertion length in the scope information S, and FIG. 7 is a diagram explaining the bending angle and the rotation amount in the scope information S.

As illustrated in FIG. 6, at a time T, the insertion section 20 is inserted into a lumen by a length a. At a time T+α, the insertion section 20 is inserted into the lumen by a length a+b. The scope information acquisition unit 42 acquires, as scope information S, a change amount of the insertion length at the time T+a with reference to the insertion length of the insertion section 20 at the time T. That is, the scope information acquisition unit 42 acquires a length b as a change amount of the insertion length as scope information S.

As also illustrated in FIG. 7, at a time T, the distal end of the insertion section 20 has a bending angle of 0°, and a rotation amount in the circumferential direction of 0. At a time T+α, the distal end of the insertion section 20 has a bending angle c and a rotation amount d in the circumferential direction. The scope information acquisition unit 42 acquires, as scope information S, change amounts of the bending angle and the rotation amount at the time T+a with reference to the bending angle and the rotation amount at the time T. In this case, the scope information acquisition unit 42 acquires c as a change amount of the bending angle and d as a change amount of the rotation amount in the circumferential direction as scope information S.

The landmark recognition unit 43 (FIG. 5) performs landmark recognition processing to recognize a landmark in an endoscopic image. Here, the landmark is a portion serving as a mark in the endoscopic image, and by tracking the landmark in time series, an operation (change amount) of the endoscopic scope 10 can be recognized. Specific examples of the landmark include a fold of the large intestine, the duodenum, or the like; a lesion such as a polyp; a start point, an end point, or an intermediate point of an organ (in the case of the large intestine, a splenic flexure, a hepatic flexure, an ileocecal portion, or the like); and the like. The landmark recognition unit 43 can recognize a landmark in an endoscopic image by various methods. For example, the landmark recognition unit 43 is constituted of a recognizer (learned model) constituted of a CNN and has undergone machine learning, and recognizes a landmark from an input endoscopic image.

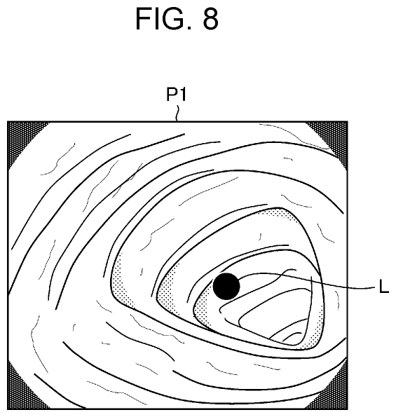

FIG. 8 is a view explaining an example of a landmark recognized by the landmark recognition unit 43.

The landmark recognition unit 43 recognizes a landmark L that is a lesion part in an intraluminal image P1. When the landmark recognition unit 43 is constituted of the recognizer, a score relating to the recognition of the landmark L may be output. This score is used as recognition reliability which will be described in a second embodiment.

The scope information correction unit 44 (FIG. 5) performs scope information correction processing to correct the scope information S using information relating to the landmark recognized by the landmark recognition unit 43. Here, the information relating to the landmark is, specifically, information relating to a temporal change in the position of the landmark recognized in each of intraluminal images P continuous in time series. The scope information correction unit 44 can correct the scope information S using the information relating to the landmark according to various aspects. For example, the scope information correction unit 44 replaces the change amount of the endoscopic scope 10 obtained based on the information relating to the landmark with the scope information acquired by the scope information acquisition unit 42 to acquire corrected scope information T.

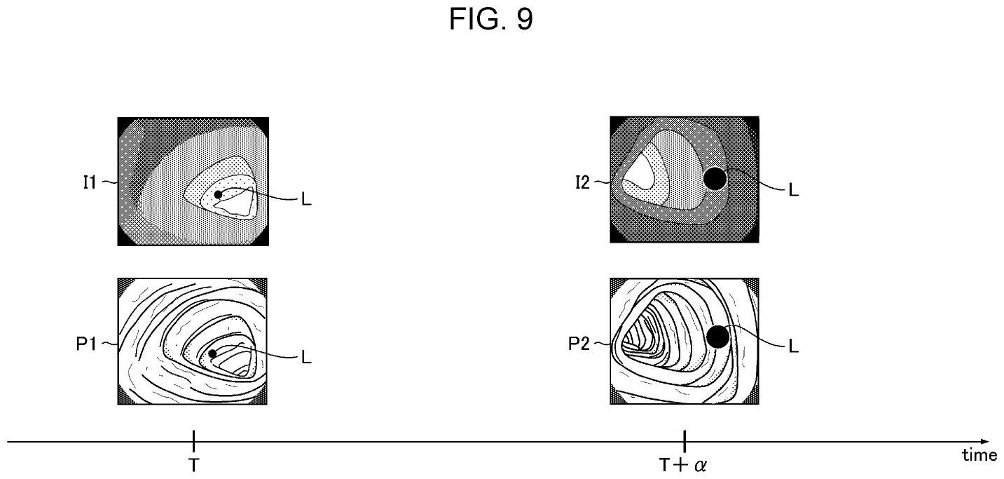

FIG. 9 is a diagram explaining information relating to a landmark acquired by the scope information correction unit 44.

As described below, the scope information correction unit 44 tracks a landmark in time series, uses depth information corresponding to the landmark as information relating to the landmark, and acquires a change amount of the endoscopic scope 10 in a lumen.

First, the landmark recognition unit 43 recognizes a landmark L in an intraluminal image P1 at a time T. The landmark recognition unit 43 also recognizes the landmark L (a correspondence point of the landmark L) in a depth image Il corresponding to the intraluminal image P1. Then, the scope information correction unit 44 acquires depth information of the landmark L at the time T.

At a time T+α, the landmark recognition unit 43 recognizes the landmark L recognized at the time T also in an intraluminal image P2. The landmark recognition unit 43 also recognizes the landmark L (a correspondence point of the landmark L) in a depth image 12 corresponding to the intraluminal image P2. Then, the scope information correction unit 44 acquires depth information of the landmark L at the time T+α. Thereafter, the scope information correction unit 44 acquires a change amount X of the insertion length of the endoscopic scope 10, acquires a change amount Y of the bending angle of the scope, and acquires a change amount Z of the rotation amount in the circumferential direction of the scope based on a temporal change of the depth information of the landmark L (the correspondence point of the landmark L) from the time T to the time T+α. In this example, the change amounts X, Y, and Z are acquired based on the temporal change in the position of the landmark L between the times T and T+α; however, the change amounts X, Y, and Z may be acquired based on the temporal change in the position of the landmark L among three or more times.

Then, the scope information correction unit 44 corrects the scope information using the change amount of the scope acquired based on the landmark L. For example, the scope information correction unit 44 replaces the scope information S acquired by the scope information acquisition unit 42 with the change amount of the endoscopic scope 10 acquired based on the landmark L. Specifically, the scope information correction unit 44 corrects the change amount b of the insertion length acquired by the scope information acquisition unit 42 to the change amount X of the insertion length based on the landmark. Also, the scope information correction unit 44 corrects the change amount c of the bending angle acquired by the scope information acquisition unit 42 to the change amount Y of the bending angle based on the landmark. Further, the scope information correction unit 44 corrects the change amount d in the circumferential direction acquired by the scope information acquisition unit 42 to the change amount Z in the circumferential direction based on the landmark.

In the above-described example, the change amount of the endoscopic scope 10 is acquired using the landmark in the depth image; however, the present invention is not limited thereto. For example, the movement of the landmark in the intraluminal image P and the change amount of the endoscopic scope 10 may be estimated through machine learning or the like. A recognizer that has undergone machine learning on the movement (change amount) of the landmark in the intraluminal image P with respect to a plurality of patterns of change amounts (translation, rotation, and bending) of the endoscopic scope 10 in advance is prepared. Then, the translation amount of the endoscopic scope 10 is calculated and estimated from the change amount of the landmark in the intraluminal image P from the time T to the time T+a by the recognizer.

The depth information acquisition unit 45 (FIG. 5) performs depth information acquisition processing to acquire depth information of the endoscopic image based on the endoscopic image and the scope information T corrected by the scope information correction unit 44. The depth information acquisition unit 45 is a learned model that is constituted of a CNN and has undergone machine learning. When the corrected scope information T (or the scope information S) and the time-series intraluminal images are input, the depth information acquisition unit 45 outputs a depth image having depth information.

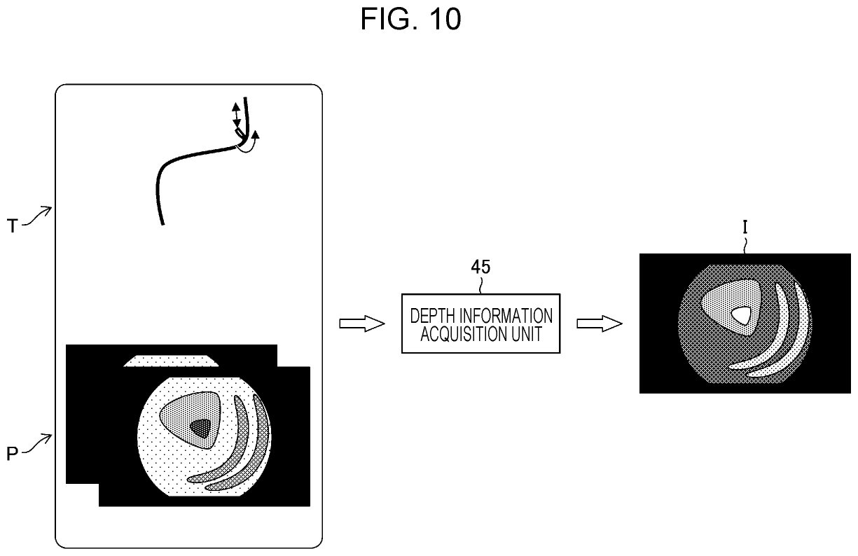

FIG. 10 is a diagram explaining acquisition of a depth image I from corrected scope information T and an intraluminal image P.

As illustrated in FIG. 10, the scope information T corrected by the scope information correction unit 44 and the intraluminal image P are input to the depth information acquisition unit 45. The scope information T corrected by the scope information correction unit 44 presents the change amount of the scope in the lumen more correctly. Accordingly, the depth information acquisition unit 45 can output a depth image I having more correct depth information.

The display control unit 46 (FIG. 5) generates image data for display based on the endoscopic image (moving image 38) acquired by the image acquisition unit 40, and outputs the image data for display to the display unit 16. Also, the display control unit 46 generates geometric information relating to the lumen and outputs the geometric information to the display unit 16. The voice control unit 47 (FIG. 5) controls voice output by a speaker 17. For example, the voice control unit 47 controls the speaker 17 to output a notification sound to the user.

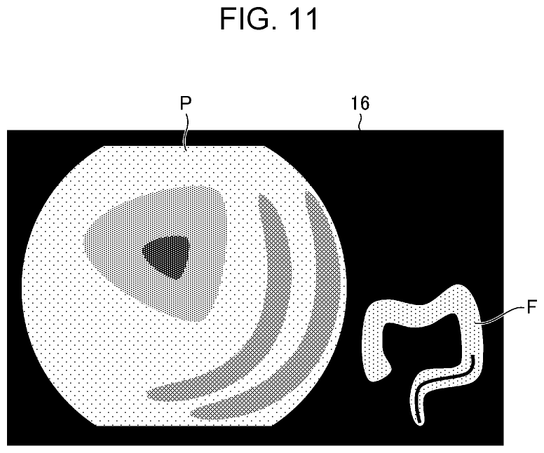

FIG. 11 is a view illustrating an example of an intraluminal image P and geometric information F of a lumen displayed on the display unit 16.

An intraluminal image P captured by the endoscope system 9 is displayed in a main region of the display unit 16. Geometric information F of the lumen is displayed in a sub-region of the display unit 16. The geometric information F of the lumen is generated by the display control unit 46 and output to the display unit 16. The geometric information F is generated based on the corrected scope information T and based on the accurate depth information acquired by the depth information acquisition unit 45. The geometric information F indicates the shape of the lumen (the shape of the large intestine) that is the observation target and the current position of the endoscopic scope 10. Also, the geometric information F may indicate the position of a lesion, the position of a treatment tool, and the like. As described above, since the depth information acquisition unit 45 acquires accurate depth information, the geometric information F using the depth information can correctly present position information or the like.

### Image Processing Method and Program

Next, an image processing method using the image processing apparatus 14 and a program for causing the image processing apparatus 14 to execute the image processing method will be described.

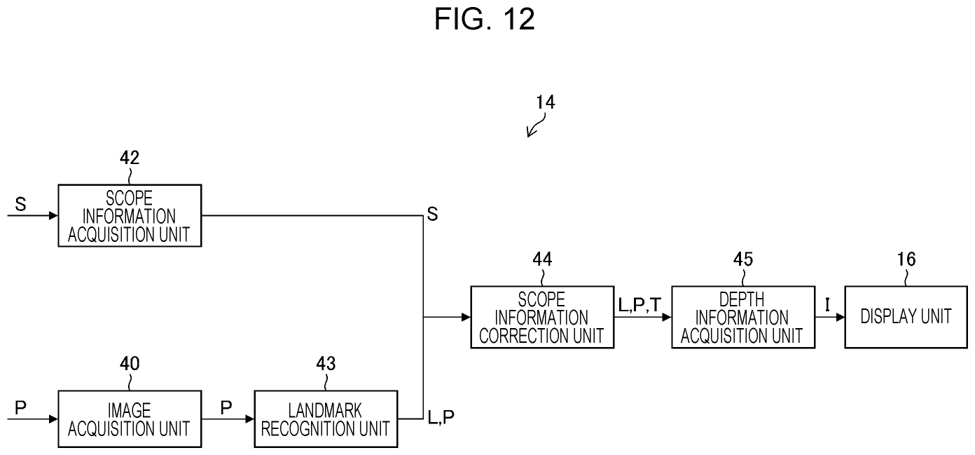

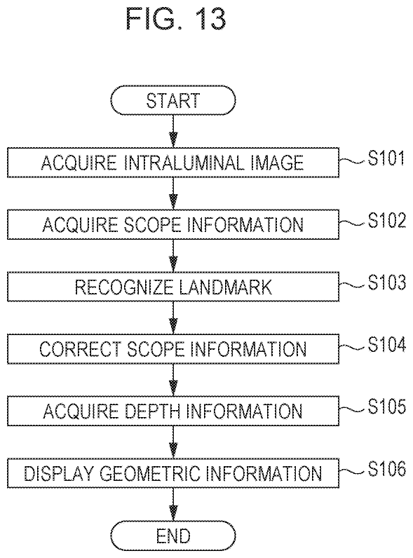

FIGS. 12 and 13 are diagrams explaining a flow of acquisition of depth information. FIG. 12 is a diagram illustrating a flow of data in a functional block diagram of the image processing apparatus 14. FIG. 13 is a flowchart presenting an image processing method using the image processing apparatus 14.

First, the image acquisition unit 40 acquires an intraluminal image P (image acquisition step: step S101). Here, the intraluminal image P is a frame image 38a constituting a moving image 38. Also, the scope information acquisition unit 42 acquires scope information S (scope information acquisition step: step S102). Next, the landmark recognition unit 43 recognizes a landmark L in the intraluminal image P (landmark recognition step: step S103). Then, the scope information correction unit 44 corrects the scope information S (scope information correction step: step S104). For example, the scope information correction unit 44 acquires scope information T that is a change amount of the endoscopic scope 10 acquired based on the landmark L. Then, the depth information acquisition unit 45 acquires a depth image I having depth information of the intraluminal image using the intraluminal image P and the scope information T (depth information acquisition step: step S105). Then, the display unit 16 displays geometric information relating to the lumen on the display unit 16 based on the depth information (display control processing step: step S106).

As described above, in the present embodiment, the landmark in the intraluminal image is recognized, and the scope information S is corrected using the information relating to the recognized landmark. Accordingly, accurate depth information of an intraluminal image can be acquired based on the corrected accurate scope information T.

In the above-described embodiment, the example in which the scope information T is acquired by correcting the scope information S acquired by the scope information acquisition unit 42 using the information relating to the landmark has been described. However, the scope information S acquired by the scope information acquisition unit 42 does not need to be corrected in some cases. That is, when correct scope information T can be obtained by performing correction in the scope information correction unit 44, it is preferable to correct the scope information S. Such an embodiment will be described below.

### Second Embodiment

Next, a second embodiment will be described. In the present embodiment, the scope information correction unit 44 corrects the scope information S in accordance with recognition reliability of the landmark.

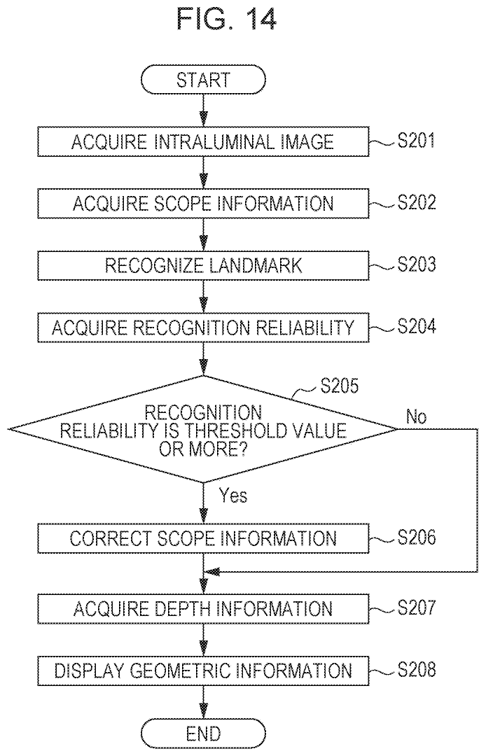

FIG. 14 is a flowchart presenting an image processing method according to the present embodiment.

First, the image acquisition unit 40 acquires an intraluminal image P (step S201). Also, the scope information acquisition unit 42 acquires scope information S (step S202). Next, the landmark recognition unit 43 recognizes a landmark L in the intraluminal image P (step S203).

Next, the landmark recognition unit 43 acquires recognition reliability of the recognized landmark (step S204). Here, the recognition reliability of the landmark is acquired by various methods. For example, the landmark recognition unit 43 is constituted of a recognizer (learned model) that has undergone machine learning, and a score obtained when the landmark is recognized can be used as the recognition reliability of the landmark.

Then, the scope information correction unit 44 determines whether the recognition reliability of the landmark is a threshold value or more (step S205). When the recognition reliability of the landmark is less than the threshold value, the scope information correction unit 44 does not correct the scope information S. In this case, the depth information acquisition unit 45 acquires depth information using scope information S of an actual measurement value that has not been corrected (step S207). In contrast, when the recognition reliability of the landmark is the threshold value or more, the scope information correction unit 44 corrects the scope information S (step S206). Then, the depth information acquisition unit 45 acquires depth information based on the corrected scope information T (step S207). Then, the display unit 16 displays geometric information relating to the lumen on the display unit 16 based on the depth information (step S208).

As described above, when the landmark recognition unit 43 correctly recognizes the landmark, the scope information correction unit 44 can correctly acquire the change amount of the endoscopic scope 10 based on the landmark. In contrast, when the landmark recognition unit 43 cannot correctly recognize the landmark, it may be difficult for the scope information correction unit 44 to correctly acquire the change amount of the endoscopic scope 10 based on the landmark. Thus, in the present embodiment, since the scope information S is corrected in accordance with the recognition reliability of the landmark, accurate depth information can be acquired.

### Third Embodiment

Next, a third embodiment will be described. In the present embodiment, scope information is corrected in accordance with a correction value obtained by the scope information correction unit 44.

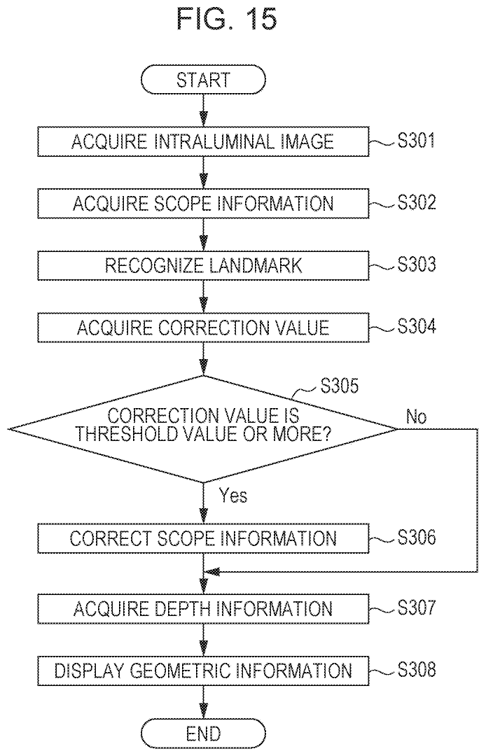

FIG. 15 is a flowchart presenting an image processing method according to the present embodiment.

First, the image acquisition unit 40 acquires an intraluminal image P (step S301). Also, the scope information acquisition unit 42 acquires scope information S (step S302). Next, the landmark recognition unit 43 recognizes a landmark L in the intraluminal image P (step S303). Next, the scope information correction unit 44 acquires a correction value (step S304).

The scope information correction unit 44 outputs the correction value obtained from information relating to the landmark. For example, as described in the first embodiment, when the information relating to the landmark is depth information corresponding to the landmark L, the scope information correction unit 44 acquires a change amount of the endoscopic scope 10 obtained based on the information relating to the landmark, and acquires the difference between the scope information T corrected with the change amount and the scope information S before the correction as a correction value. Then, the scope information correction unit 44 determines whether the correction value is a threshold value or more (step S305). When the correction value is less than the threshold value, the scope information correction unit 44 does not correct the scope information S, and the depth information acquisition unit 45 acquires depth information (step S307). In contrast, when the correction value is the threshold value or more, the scope information correction unit 44 corrects the scope information S (step S306), and the depth information acquisition unit 45 acquires depth information (step S307). Then, the display unit 16 displays geometric information relating to the lumen on the display unit 16 based on the depth information (step S308).

As described above, when the correction value is the threshold value or more, the difference between the scope information S that is the actual measurement value and the scope information T corrected with the change amount of the endoscopic scope 10 acquired based on the landmark L is large. Thus, the scope information correction unit 44 performs correction on the scope information S. In contrast, when the correction value is less than the threshold value, the difference between the scope information S that is the actual measurement value and the scope information T corrected with the change amount of the endoscopic scope 10 acquired based on the landmark L is small. Thus, even though the scope information S is used as it is, the influence on the accuracy of the depth information is small, and hence the scope information correction unit 44 does not correct the scope information S. Accordingly, in the present embodiment, since the scope information is corrected in accordance with the correction value, accurate depth information can be efficiently acquired.

### Others

In the above-described embodiment, the hardware structures of processing units that execute various kinds of processing (for example, the image acquisition unit 40, the scope information acquisition unit 42, the landmark recognition unit 43, the scope information correction unit 44, the depth information acquisition unit 45, the display control unit 46, and the voice control unit 47) are various processors as described below. The various processors include a central processing unit (CPU) that is a general-purpose processor that executes software (program) to function as various processing units; a programmable logic device (PLD) that is a processor whose circuit configuration can be changed after manufacture, such as a field programmable gate array (FPGA); a dedicated electric circuit that is a processor having a circuit configuration designed exclusively for executing specific processing, such as an application specific integrated circuit (ASIC); and the like.

One processing unit may be constituted of one of these various processors, or may be constituted of two or more processors of the same type or different types (for example, a plurality of FPGAs or a combination of a CPU and an FPGA). Alternatively, a plurality of processing units may be constituted of one processor. As an example in which the plurality of processing units are constituted of one processor, first, there is an embodiment in which one processor is constituted of a combination of one or more CPUs and software, and the processor functions as the plurality of processing units, as typified by a computer such as a client or a server. Second, there is an embodiment of using a processor that implements the functions of the entire system including a plurality of processing units by one integrated circuit (IC) chip, as typified by a system on chip (SoC) or the like. As described above, the various processing units are constituted using one or more of the above-described various processors as the hardware structures.

Further, more specifically, the hardware structures of these various processors are an electric circuit (circuitry) obtained by combining circuit elements such as semiconductor elements.

Each of the above-described configurations and functions can be appropriately implemented by any hardware, software, or a combination of both. For example, the present invention can be applied to a program for causing a computer to execute the above-described processing steps (processing procedures), a computer-readable recording medium (non-transitory recording medium) having such a program recorded therein, or a computer capable of installing such a program.

Although the examples of the present invention have been described above, the present invention is not limited to the above-described embodiments, and of course various modifications can be made without departing from the spirit of the present invention.

### REFERENCE SIGNS LIST

9 endoscope system
10 endoscopic scope
11 light source device
12 endoscope processor device
13 display device
14 image processing apparatus
15 operating unit
16 display unit
17 speaker
20 insertion section
21 hand operation section
22 universal cord
24 rotation scale
25 soft part
26 bending part
27 tip part
28 imaging element
29 bending operation knob
30 air/water supply button
31 suction button
32 still image capturing instruction portion
33 treatment tool lead-in port
34 length scale
35 light guide
36 signal cable
37a connector
37b connector
40 image acquisition unit
41 CPU
42 scope information acquisition unit
43 landmark recognition unit
44 scope information correction unit
45 depth information acquisition unit
46 display control unit
47 voice control unit
48 memory

What is claimed is:

1. An image processing apparatus comprising a processor,
wherein the processor performs:
image acquisition processing of acquiring a time-series intraluminal image captured by a scope of an endoscope;
scope information acquisition processing of acquiring scope information relating to a change of the scope;
landmark recognition processing of recognizing a landmark in the intraluminal image;
scope information correction processing of correcting the scope information using information relating to the landmark recognized in the landmark recognition processing; and
depth information acquisition processing of acquiring depth information of the intraluminal image using the intraluminal image and the scope information corrected in the scope information correction processing.

2. The image processing apparatus according to claim 1, wherein, with reference to a position of the scope at a time T, the scope information acquisition processing acquires a change amount of an insertion length of the scope and change amounts relating to bending and rotation of the scope at a time T+α.

3. The image processing apparatus according to claim 1, wherein the scope information acquisition processing acquires information relating to an insertion length of the scope and bending and rotation of the scope from an operation of an operation section of the scope.

4. The image processing apparatus according to claim 1,
wherein the landmark recognition processing recognizes a temporal change of a correspondence point of the landmark, and
wherein the scope information correction processing corrects the scope information using the temporal change of the correspondence point.

5. The image processing apparatus according to claim 1,
wherein the landmark recognition processing outputs recognition reliability of the recognized landmark, and
wherein the scope information correction processing determines whether to execute the correction of the scope information based on the recognition reliability, and performs the correction based on a result of the determination.

6. The image processing apparatus according to claim 1, wherein the scope information correction processing outputs a correction value obtained from the information relating to the landmark, determines whether to execute the correction based on the correction value, and performs the correction based on a result of the determination.

7. The image processing apparatus according to claim 1,
wherein the processor performs:
display control processing of displaying geometric information relating to a lumen on a display unit based on the depth information acquired in the depth information acquisition processing.

8. The image processing apparatus according to claim 7, wherein the geometric information is at least one of a shape of the lumen, a position of a lesion, a position of the scope, or a position of a treatment tool.

9. An image processing method using an image processing apparatus comprising a processor, the method, performed by the processor, comprising:
an image acquisition step of acquiring a time-series intraluminal image captured by a scope of an endoscope;
a scope information acquisition step of acquiring scope information relating to a change of the scope;
a landmark recognition step of recognizing a landmark in the intraluminal image;
a scope information correction step of correcting the scope information using information relating to the landmark recognized in the landmark recognition step; and
a depth information acquisition step of acquiring depth information of the intraluminal image using the intraluminal image and the scope information corrected in the scope information correction step.

10. A non-transitory, computer-readable tangible recording medium having recorded therein a program for causing, when read by a computer, a processor of the computer to execute the image processing method according to claim 9.
